(19) United States
(12) Patent Application Publication
(10) Pub. No.: US 2023/0146194 A1
(43) Pub. Date: May 11, 2023

(54) DENTAL MIRROR STRUCTURE CAPABLE OF SPRAYING AIR

(71) Applicant: ACME TOOLING TECHNOLOGY CO., LTD., CHANGHUA COUNTY (TW)

(72) Inventors: KUO FANG WANG, CHANGHUA COUNTY (TW); TE KUAN, CHANGHUA COUNTY (TW)

(21) Appl. No.: 17/521,119

(22) Filed: Nov. 8, 2021

(51) Int. Cl. A61B 1/247; A61B 1/012; A61B 1/00; A61C 17/022

(52) U.S. Cl. CPC A61B 1/247; A61B 1/012; A61B 1/00128; A61C 17/022

(57) ABSTRACT

A dental mirror structure contains a body and a connector. The body includes an inspector, a connection segment, and a first air conduit. The inspector includes a lens, and the first air conduit has an inlet and an outlet adjacent to the lens. The connector is hollow and columnar, and the connector includes a second air conduit defined on a center thereof, a connection portion extending from a first end of the connector, a first stem extending from the connection portion, a second stem extending from a second end of the connector, and a fixing extension extending from the second end of the connector and surrounding the second stem. Thereby, the first stem is connected with an air supply element, the second stem is fitted with the inlet the body, and the fixing extension is connected with the connection segment.

### FIELD OF THE INVENTION

The present invention relates to a dental mirror structure which sprays air to clean teeth and is connected and removed quickly.

### BACKGROUND OF THE INVENTION

A conventional dental mirror is applied to push tongue or cheeks so as to inspect teeth. The conventional dental mirror includes a grip and an inspector, and a spray gun is configured to spray air, water or mist to clean teeth.

However, the spray gun is connected with an air hose fixed with a treatment table, thus connecting or removing the spray gun troublesomely.

The present invention has arisen to mitigate and/or obviate the afore-described disadvantages.

### SUMMARY OF THE INVENTION

The primary aspect of the present invention is to provide a dental mirror structure which contains the body having the first air conduit so that when the air supply element is connected with the connector, the body sprays the air to clean the teeth.

Another aspect of the present invention is to provide a dental mirror structure which contains the fixing extension of the connector connected with or removed from the connection segment so as to connect or remove the body with or from the air supply element quickly.

To obtain above-mentioned aspects, a dental mirror structure provided by the present invention contains: a body and a connector.

The body is hollow and is formed in a post shape, the body includes an inspector formed on a first end of the body, the body includes a connection segment formed on a second end of the body, and the inspector includes a lens. The body further includes a first air conduit passing through a center of the body, the first air conduit has an inlet defined in a first end of the connection segment, and the first air conduit further has an outlet adjacent to the lens.

The connector is hollow and columnar, and the connector includes a second air conduit defined on a center of the connector, a first stem extending from first end of the connector, a second stem extending from a second end of the connector, and a fixing extension extending from the second end of the connector and surrounding the second stem. The first stem is connected with an air supply element, the second stem is fitted with the inlet the body, and the fixing extension is connected with the connection segment.

Preferably, the connection segment includes a coupling notch defined on a second end thereof away from the inspector, the fixing extension has at least one wing, and a respective wing of the at least one wing has a tab extending from an inner wall thereof so as to engaged with the coupling notch,

Preferably, the connection segment includes a coupling notch defined on a second end thereof away from the inspector, and the connection segment includes an inclined face formed above the coupling notch.

Preferably, the body is one-piece formed with the inspector.

Preferably, the body is made of plastic.

Preferably, the connector includes a connection portion extending from the first end thereof, and the connector includes the first stem extending from the connection portion, such that the air supply element is connected with the first stem, and the air supply element includes a defining sleeve fitted with the connection portion of the connector.

Preferably, the body is detachably connected with the inspector, and the inspector includes the lens and a column in which a third air conduit is defined, wherein the body includes a locating segment extending from an end of the column away from the lens, the locating segment has an air inflow portion communicating with the third air conduit, and an opening is defined between the third air conduit and the lens, and the locating segment is connected with the first end of the body away from the inlet.

Preferably, the inflow portion has a beveled or an arcuate surface formed thereon.

Preferably, the locating segment is fitted with the first end of the body away from the inlet.

Preferably, the locating segment is screwed with the first end of the body away from the inlet.

Preferably, the connector is engaged with and is rotated relative to the body, such that the air supply element is operated smoothly. The body is made of the plastic so as to be compact.

The body is detachably connected with the inspector, for example, the column of the inspector is detachably screwed or is engaged (not shown) with the body, thus removing the inspector to be cleaned easily. In addition, the inflow portion has the beveled or then arcuate surface formed thereon so as to flow the air easily.

### BRIEF DESCRIPTION OF THE DRAWINGS

FIG. 1 is a perspective view showing the assembly of a dental mirror structure according to a first embodiment of the present invention.

FIG. 2 is a perspective view showing the exploded components of the dental mirror structure according to the first embodiment of the present invention.

FIG. 3 is a cross sectional view showing the assembly of the dental mirror structure according to the first embodiment of the present invention.

FIG. 4 is a perspective view showing the assembly of a dental mirror structure according to a second embodiment of the present invention.

FIG. 5 is a perspective view showing the exploded components of the dental mirror structure according to the second embodiment of the present invention.

FIG. 6 is a cross sectional view showing the assembly of the dental mirror structure according to the second embodiment of the present invention.

### DETAILED DESCRIPTION OF THE PREFERRED EMBODIMENTS

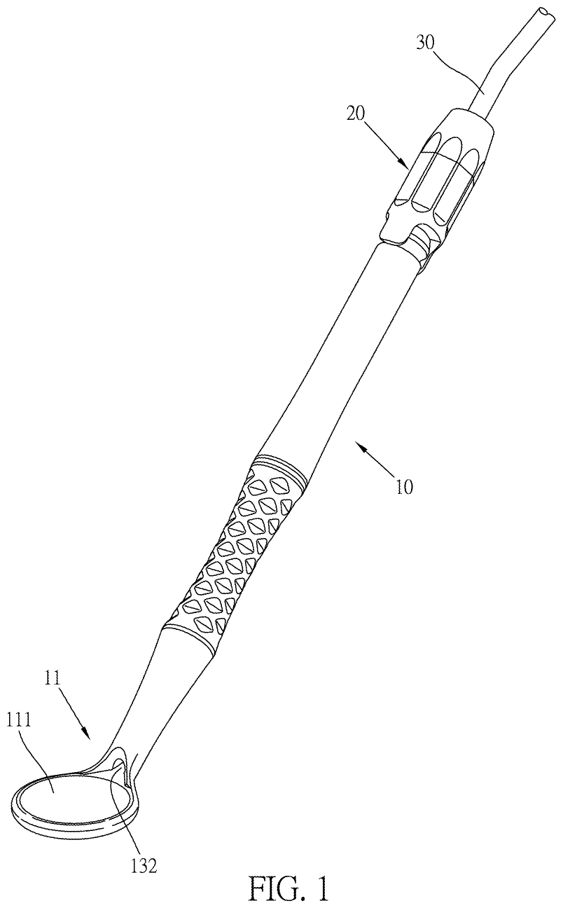

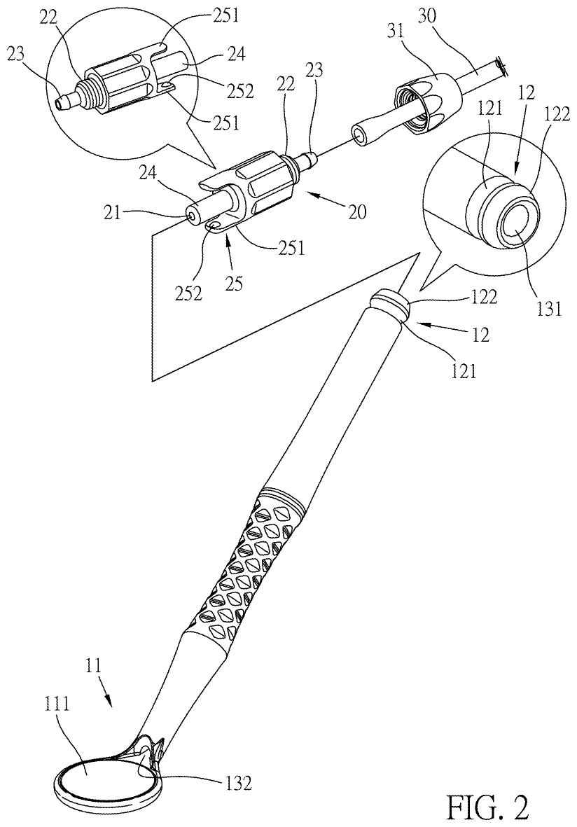

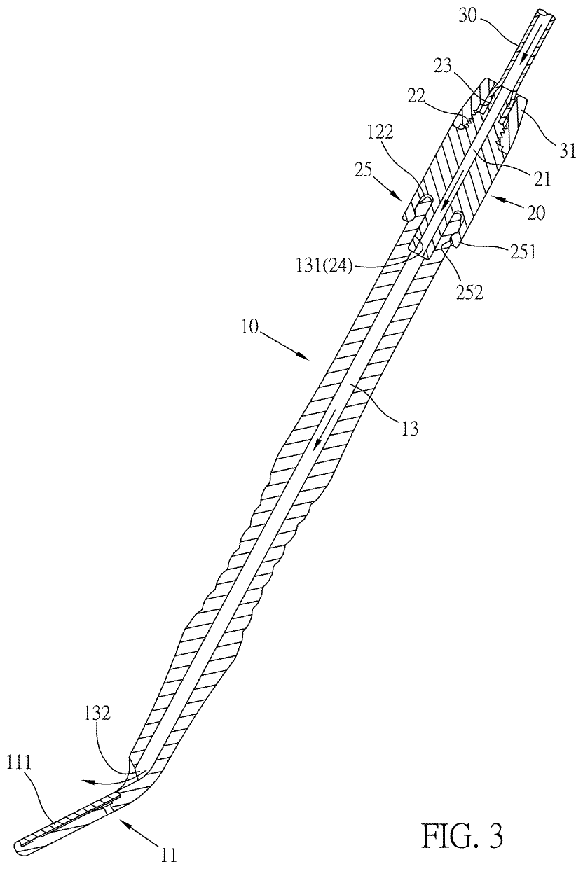

With reference to FIGS. 1-3, a dental mirror structure according to a first embodiment of the present invention comprises: a body 10, a connector 20, and an air supply element 30.

The body 10 is hollow and is formed in a post shape, the body 10 includes an inspector 11 formed on a first end thereof, and the body 10 includes a connection segment 12 formed on a second end thereof, the inspector 11 includes a lens 111, and an angle is defined between the lens 111 and the body 10 so that the inspector 11 inspects teeth by ways of the lens 111. The body 10 further includes a first air conduit 13 passing through a center thereof, and the first air conduit 13 has an inlet 131 defined in a first end of the connection segment 12, the first air conduit 13 further has an outlet 132 adjacent to the lens 111. The connection segment 12 includes a coupling notch 121 defined on a second end thereof away from the inspector 11, and the connection segment 12 includes an inclined face 122 formed above the coupling notch 121. The body 10 is made of plastic and is one-piece formed with the inspector 11.

The connector 20 is hollow and columnar, and the connector 20 includes a second air conduit 21 defined on a center thereof, a connection portion 22 extending from a first end of the connector 20, a first stem 23 extending from the connection portion 22, a second stem 24 extending from a second end of the connector 20, and a fixing extension 25 extending from the second end of the connector 20 and surrounding the second stem 24, wherein the fixing extension 25 has two opposite wings 251, and a respective wing 251 has a tab 252 extending from an inner wall thereof.

The air supply element 30 is connected with the first stem 23 and includes a defining sleeve 31 fitted with the connection portion 22 of the connector 20, thus fixing the sir supply element 20 and the air supply element 30 securely. The second stem 24 is fitted with the inlet 131 of the body 10, a tab 252 of the respective wing 251 of the fixing extension 25 is engaged with the coupling notch 121, and the inclined face 122 facilitates an engagement of the tab 252 and the notch 121, thus connecting the connector 20 and the body 10 quickly. Thereby, air is fed into the first air conduit 13 from the second air conduit 21 and is supplied to the teeth from the outlet 132 via the inspector 11, thus cleaning the teeth. To remove the connector 20 from the body 10, the respective wing 251 of the fixing extension 25 is detachable from the notch 121 so that the second stem 24 of the connector 20 is removed from the body 10, thus removing the connector from the body quickly.

Accordingly, the body 10 includes the first air conduit 13 and the connection segment 12 so that the air supply element 30 is connected with the connector 20, and the body 10 sprays the air to clean the teeth. Preferably, the connector 20 is detachable from the body 10 quickly.

In assembly, the air supply element 20 is connected with the first stem 23 of the connector 20, the defining sleeve 31 is fixed with the connection portion 22, and the second stem 24 of the connector 20 is fitted with the inlet 131 of the body 10, the tab 252 of the respective wing 251 of the fixing extension 25 is engaged with the coupling notch 121, thus connecting the connector 20 with the body 10 quickly. After the tab 252 of the respective wing 251 is removed from the notch 121, the connector 20 is detachable from the body 10 easily.

In application, the air is fed into the second air conduit 21 via the first stem 23 of the connector 20 and flows into the first air conduit 13 via the inlet 131 of the body 10, and the air is sprayed to the teeth from the outlet 132 via the inspector 11, thus cleaning the teeth.

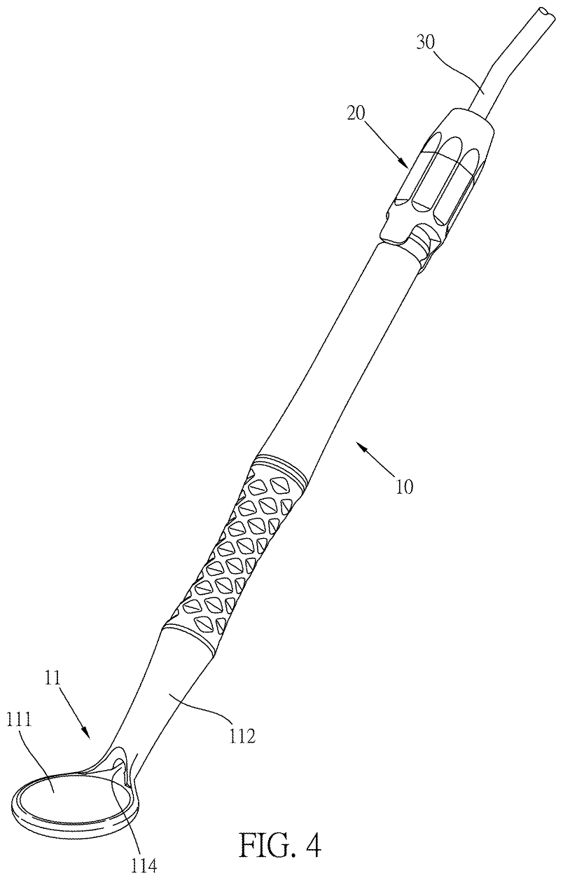

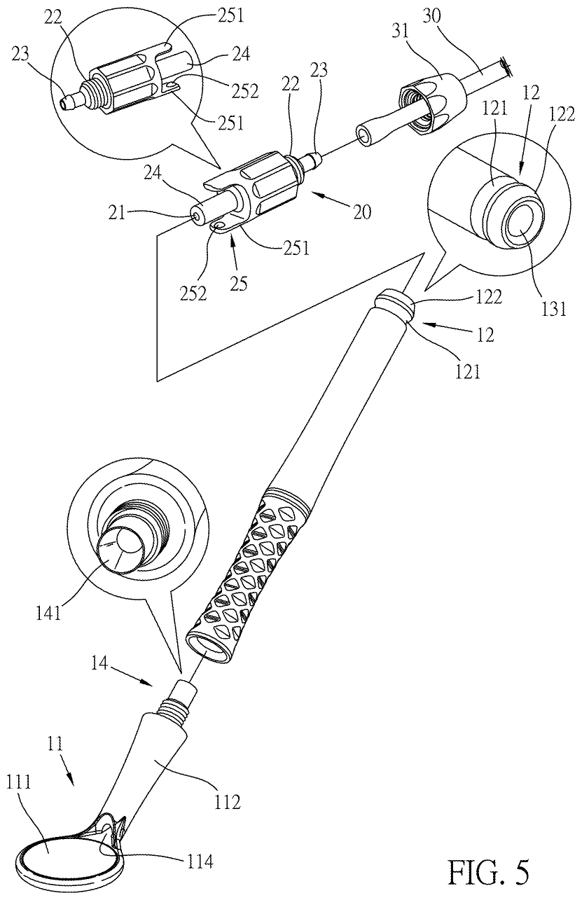

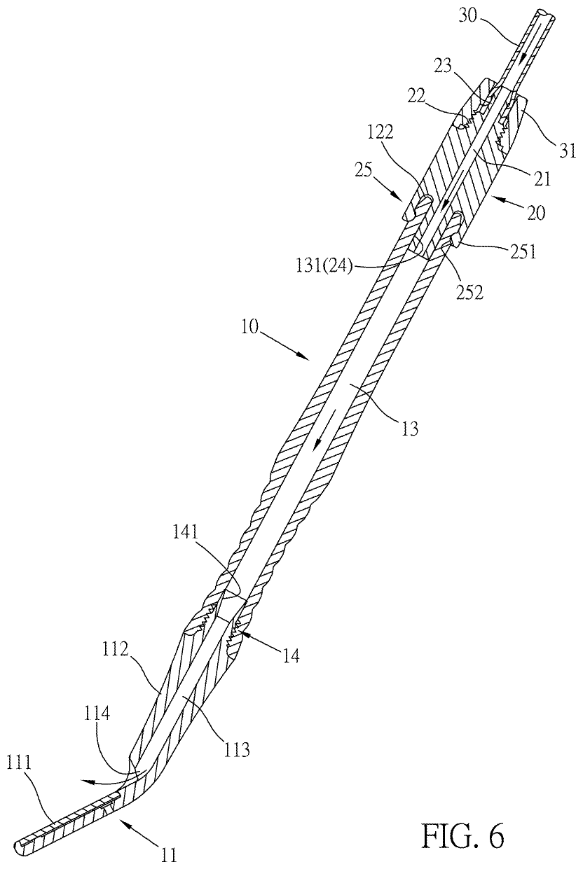

Referring to FIGS. 4-6, in a second embodiment, the body 10 is removably connected with the inspector 11, wherein the inspector 11 includes a lens 111 and a column 112 in which a third air conduit 113 is defined, and an angle is defined between the lens 111 and the column 112, wherein the body 10 includes a locating segment 14 extending from an end of the column 112 away from the lens 111, and the locating segment 14 has an air inflow portion 141 communicating with the third air conduit 113, wherein the inflow portion 141 has a beveled or an arcuate surface formed thereon so as to flow the air easily, and an opening 114 is defined between the third air conduit 113 and the lens 111, wherein the locating segment 14 is fitted with or is screwed with the first end of the body 10 away from the inlet 131, thus fixing the inspector 11 easily and cleaning the teeth.

Accordingly, the body 10 of the dental mirror structure includes the first air conduit 13 so that when the air supply element 30 is connected with the connector 20, the body 10 sprays the air to clean the teeth. Preferably, the connector 20 is detachable from the body 10 quickly. Furthermore, the fixing extension 25 of the connector 20 is connected with or is removed from the connection segment 12 so as to connect or remove the body 10 with or from the air supply element 30 quickly.

Preferably, the connector 20 is engaged with and is rotated relative to the body 10, such that the air supply element 30 is operated smoothly. The body 10 is made of the plastic so as to be compact.

The body 10 is detachably connected with the inspector 11, for example, the column 112 of the inspector 11 is detachably screwed or is engaged (not shown) with the body 10, thus removing the inspector to be cleaned easily. In addition, the inflow portion 141 has the beveled or then arcuate surface formed thereon so as to flow the air easily.

While the preferred embodiments of the invention have been set forth for the purpose of disclosure, modifications of the disclosed embodiments of the invention as well as other embodiments thereof may occur to those skilled in the art. Accordingly, the appended claims are intended to cover all embodiments which do not depart from the spirit and scope of the invention.

What is claimed is:

1. A dental mirror structure comprising:
a body being hollow and formed in a post shape, the body including an inspector formed on a first end of the body, the body including a connection segment formed on a second end of the body, and the inspector including a lens, the body further including a first air conduit passing through a center of the body, the first air conduit having an inlet defined in a first end of the connection segment, and the first air conduit further having an outlet adjacent to the lens;
a connector being hollow and columnar, and the connector including a second air conduit defined on a center of the connector, a first stem extending from first end of the connector, a second stem extending from a second end of the connector, and a fixing extension extending from the second end of the connector and surrounding the second stem, wherein the first stem is connected with an air supply element, the second stem is fitted with the inlet the body, and the fixing extension is connected with the connection segment.

2. The dental mirror structure as claimed in claim 1, wherein the connection segment includes a coupling notch defined on a second end thereof away from the inspector, the fixing extension has at least one wing, and a respective wing of the at least one wing has a tab extending from an inner wall thereof so as to engaged with the coupling notch.

3. The dental mirror structure as claimed in claim 1, wherein the connection segment includes a coupling notch defined on a second end thereof away from the inspector, and the connection segment includes an inclined face formed above the coupling notch.

4. The dental mirror structure as claimed in claim 1, wherein the body is one-piece formed with the inspector.

5. The dental mirror structure as claimed in claim 1, wherein the body is made of plastic.

6. The dental mirror structure as claimed in claim 1, wherein the connector includes a connection portion extending from the first end thereof, and the connector includes the first stem extending from the connection portion, such that the air supply element is connected with the first stem, and the air supply element includes a defining sleeve fitted with the connection portion of the connector.

7. The dental mirror structure as claimed in claim 1, wherein the body is detachably connected with the inspector, and the inspector includes the lens and a column in which a third air conduit is defined, wherein the body includes a locating segment extending from an end of the column away from the lens, the locating segment has an air inflow portion communicating with the third air conduit, and an opening is defined between the third air conduit and the lens, and the locating segment is connected with the first end of the body away from the inlet.

8. The dental mirror structure as claimed in claim 7, wherein the inflow portion has a beveled or an arcuate surface formed thereon.

9. The dental mirror structure as claimed in claim 7, wherein the locating segment is fitted with the first end of the body away from the inlet.

10. The dental mirror structure as claimed in claim 7, wherein the locating segment is screwed with the first end of the body away from the inlet.
